(19) United States
(12) Patent Application Publication
(10) Pub. No.: US 2006/0009686 A1
(43) Pub. Date: Jan. 12, 2006

(54) Dermatoscopy positioning apparatus for the head

(76) Inventor: Alex Boukas, Mattituck, NY (US)

Correspondence Address: BODNER & O'ROURKE, LLP, 425 Broadhollow Road, Ste. 108, Melville, NY, 11747, US

(21) Appl. No.: 11/175,901

(22) Filed: Jul. 6, 2005

Related U.S. Application Data
(60) Provisional application No. 60/585,756, filed on Jul. 6, 2004.

(51) Int. Cl. A61B 5/00

(52) U.S. Cl. 600/315

(57) ABSTRACT

An apparatus for examining patients for nevi is described. The apparatus includes a head piece having at least one positioning means for positioning a patient's head in a reproducible position from one examination to another. The head piece further includes a camera that is connected to the head piece that can take a picture of at least a portion of a patient's head at a first examination and substantially the same portion of a patient's head at a second examination. The position of the camera can be set so that the picture taken during the first examination can be accurately compared to the picture taken during the second examination.

This is a conversion of provisional patent application Ser. No. 60/585,756 filed on Jul. 6, 2004, the disclosures of which are incorporated herein by reference.

### FIELD OF THE INVENTION

The present invention relates to improvements in skin cancer detecting devices.

### BACKGROUND OF THE INVENTION

Everyone who is exposed to the sun runs the risk of developing melanoma. Exposure to cigarette smoke, certain types of building materials and other chemicals increases the danger of developing a malignant melanoma. Left unchecked a malignant melanoma will proliferate and grow, eventually killing the host.

Squamous cells are skin cells that make up most of the outer layer; basal cells are found at the bottom of the skin's outer layer (the epidermis). Cancer of either of these types of skin cells is known as a carcinoma. While melanoma is a cancer of the melanocytes, basal cell carcinoma (BCC) and squamous cell carcinoma (SCC) are cancers of the keratinocyte—the skin cells that distribute the melanin released by melanocytes. While usually not fatal, BCC and SCC can spread to other parts of the body and cause serious health consequences.

Both BCC and SCC occur almost exclusively in skin that was exposed to the sun, usually in people with fair skin. They usually do not appear until adulthood, as they are the result of accumulative damage from UV radiation. If discovered and treated early, 95% of all BCC and SCC cases are cured.

Basal cell carcinoma (BCC) is the most common type of skin cancer in the United States, 9 diagnosed in 800,000 people each year. It has traditionally been seen most commonly in the elderly, but the age of onset is steadily decreasing. BCC tumors usually appear as small, fleshy bumps, often on the head or neck. They can, however, appear in other areas of the body as well (usually sun-exposed areas). BCC can often resemble other skin conditions such as psoriasis and eczema, so it is important to have any skin problem examined by a dermatologist. BCC tumors grow slowly and rarely metastasize; however, if unchecked, they can grow downward into the bone and cause serious damage.

SCC is the second most common skin cancer in the United States, affecting more than 200,000 people every year. Like BCC, it is usually caused by chronic exposure to sunlight and other sun damage is usually present in an area that develops SCC. However, SCC can also occur in areas of the skin that have been damaged by burns, scars or chemicals. People with medical conditions that produce chronic skin inflammation or suppress the immune system are at increased risk for SCC. Occasionally it also occurs in skin that has not been exposed to the sun; the tendency to develop this condition may be hereditary.

SCC tumors appear as nodules or red, scaly patches of skin, most often on the rim of the ear and the lower lip. They can also appear as a wart-like growth that crusts and bleeds occasionally or open sore that won't heal. Tumors can grow into masses that cover large areas of the body. Unlike BCC, SCC is capable of metastasizing to other areas of the body. Metastasis occurs only in a small percentage of cases, but if SCC spreads to other tissues and organs, it can be fatal.

Melanoma is the most common fatal skin cancer and it is also one of the most common skin cancers in young adults. Melanoma often occurs at a younger age than BCC or SCC because it is caused by intermittent intense exposures to UV radiation, as opposed to cumulative damage over a lifetime. Melanoma occurs when melanocytes damaged by UV radiation begin to divide out of control. Keratinocytes (which are responsible for BCC and SCC) undergo the most damage when they are repeatedly exposed to low doses of UV radiation—for example, in people who develop a tan. Melanocytes, on the other hand, are most harmed by occasional exposures, such as the kind that cause a sunburn. These differences explain why BCC and SCC are usually found on chronically sun-exposed areas (such as the face, the forearms and the backs of the hands) while melanoma usually occurs on areas not often exposed to the sun (such as the back in men and the lower legs in women). Melanoma rarely occurs on the face, hands or forearms.

The risk of melanoma is doubled in people who have had five or more severe sunburns (especially burns that blistered) during adolescence. Fair-skinned people (especially those with freckles) are at the highest risk for melanoma and the risk for whites is also affected by latitude (the closer to the equator, the greater the risk). Blacks and Asians tend to develop melanoma in areas of the body not exposed to the sun—such as the nail beds and the soles of the feet. Another strong risk factor for melanoma is the presence of dysplastic nevi (abnormal or unusual moles). Not all dysplastic nevi will become malignant and researchers have not been able to figure out why having these moles increases the risk of melanoma. Melanoma does tend to run in families, so if two or more family members develop the disease, first-degree relatives (parents, siblings and children over the age of 10) should be screened for dysplastic nevi or other evidence of melanoma. Melanoma lesions usually resemble a mole, but one that is asymmetrical, has irregular shape or borders, has varying colors and is larger than 6 mm (about the size of a pencil eraser). However, while most melanomas fit this description, not all do.

Early detection of melanoma is the key part of treatment. A major factor which affects a patient's prognosis is the thickness of the melanoma, if it is removed while it is thin the patient has a much better chance for survival. Cutaneous surveillance of patients greatly increase the chance that a melanoma will be detected before it becomes deadly. The patients at greatest risk of developing melanoma have dysplastic nevi (atypical moles), many ordinary nevi, and a personal or family history of melanoma. Not all nevi will develop into melanoma however, some nevi are benign and removing them exposes the host to unnecessary surgery. Cutaneous surveillance is achieved by asking the patient if they have any moles which have been growing atypically, making a chart of the location of all nevi on the patient, or by photographically documenting the location of the nevi on the patient. Human memory is fallible and patients often times cannot remember, or never thought to check for dysplastic nevi on their body. This can lead to melanomas that should be excised being ignored or unnecessary surgery being performed to remove nevi which are not problematic. Making a chart of nevi is a useful tool for documenting the occurrence and size of nevi on a patient's body however if the patient has a large number of nevi it may be difficult to meaningfully document each one. Charts can be misread and misinterpreted thus allowing mistakes to be made regarding whether or not a nevi should be excised. Photographic documentation is a more effective way to create a record of the location of a patient's nevi however this method is not perfect either. A set of photos can help by serving as a visual record of moles, thereby allowing future determinations as to the presence or absence of a mole, or the size of color or other key traits of an existing mole compared to those traits in the initial visit. Photographs can be mislabeled and if the photograph does not have any points of reference it is difficult for the doctor to correlate the picture of the mole to the mole's position on the patient.

An example of a device for taking patient pictures is taught in U.S. Pat. No. 6,584,171 to Suzuki, et al. for an X-ray imaging detector and x-ray imaging apparatus. In that patent, the device uses an X-ray emitter and an X-ray sensitive detector to create an image of the internal workings of a human skull. In U.S. Pat. No. 6,584,171 the X-ray radiation source must be positioned at the opposite side of the patient so that the X-rays may pass through the patient and be absorbed by the X-ray detector whereas in the current invention emits a lower power form of radiation or ambient light and records the radiation reflecting off the surface of the patient's skin.

### OBJECTS OF THE INVENTION

It is an object of the present invention to provide a device that will permit accurate cutaneous surveillance of a patient.

It is an object of the present invention to provide an apparatus for accurate analysis of dysplastic nevi change on a patient.

It is an object of the present invention to provide a device and method that can help detect skin cancer by positioning the head of a patient in a reproducible position and digitally photographing the patient.

It is an object of the present invention to provide a device and method that will make it easier for physicians to detect dysplastic nevi before they endanger the life of the patient.

It is an object of the present invention to provide a device and method which will significantly reduce the number of unnecessary excisions of benign lesions.

### SUMMARY OF THE INVENTION

The present invention is directed toward to an apparatus and method for accurate cutaneous surveillance of nevi on the epidermis of a patient. The present invention is primarily useful for the early identification of dysplastic nevi and the like. The ability to reproduce a given position on the body of the patient increases the utility of the device by making it easier for the physician to correlate the position of nevi which have a possibility of being melanoma. By using the apparatus of the present invention, the user can replicate a position on the body of a patient, usually the head or face so that changes in the size and shape of a nevi can be determined from one examination to the next. The photographs can be analyzed usually digitally or by other means to determine what differences, if any, there are between photos taken on one date and photos taken on another date. The comparison will alert the physician that there may have been changes to the nevi's size and shape. This change in size or shape, etc. can be a signal that there is cancerous growth.

In a preferred embodiment of the invention the device comprises one or more or preferably multiple positioners. The positioners are members that are used to position the device in a fixed reproducible arrangement on the head of a patient. Two positioners are placed on landmarks on the side of the head such as behind the ears, in the ears, etc., and a third may be placed on the front of the head in a position such as at the bridge of the nose. This arrangement permits the device to be positioned at the same spot again in a later examination. The device has a camera which records images of the patient positioned within the device. This camera may be stationary or more preferably is movable along, for example, a track so that a number of photos of different areas on a patient may be taken by the device in a sitting.

The device may preferably have a headpiece which fits over or around at least a portion of the head of a patient. Extending from the headpiece are at least two positioning members which are preferably fixed to the headpiece. The positioning members are used to line up the apparatus of the present invention in a given configuration on the head of the patient so that when the camera takes a picture of a nevi at one time, the device can be repositioned in substantially the same position when the patient is re-checked at a later date. The positioning members may be arms that extend downwardly and can, for example, be placed behind the ears of the patient as a reference point for the first picture.

Alternatively, the device may have a single positioning member in the front of the device that is placed over the nose or the bridge of the nose to provide a reference point for the positioning of the device. In a preferred embodiment, both of the positioner members that are on the sides of the device as well as the front positioner are present to provide an accurate reference point for the taking of photographs of the nevi.

In a still further embodiment, the patient's chin may be placed on a chin rest. The chin rest can be wall mounted or mounted to the headpiece. There may be one or more additional positioning members if desired. The patient's chin on the chin rest fixes the head in a position. A camera can take pictures around the head to provide a base for comparison.

The camera that takes the photographs is preferably movable. The camera is preferably secured to a headpiece, to a wall or to some other portion of the device. The camera can, for example, be movable in a track in the headpiece. The camera may be on an arm that extends from the headpiece or from a wall or stand. The camera or the arm is preferably adjustable so that the camera can be positioned properly about the patient. The arm and the track on which the arm or camera travels is preferably calibrated so that the technician can record the settings where the camera is when pictures of the nevi are being taken.

Although the apparatus of the present invention is shown in the form of a headpiece, it will be appreciated by those skilled in the art that the headpiece can be modified to go about other portions of the body so that accurate photos of skin conditions can be taken there as well. Thus, the device may be wall mounted instead of on a headpiece. In another embodiment, the device may be mounted on a suitable stand or elsewhere as is suitable.

The positioning means can be placed behind the ears or more preferably the positioning means may have a plug on an inner surface thereof. The plug is placed in the ear so that the position of the device is fixed vis-à-vis the patient's head. The plug is preferably removable so that it can be replaced for hygienic purposes for each patient.

The positioning means may be fixed on the headpiece. However, because the size of the patient's head and the location of the ears can vary slightly from person to person, the headpiece is preferably adjustable to accommodate various sizes. In addition, the positioning members can also be movable for proper location on the patient. The headpiece and the positioning members can be calibrated so that their location or position can be recorded so that when the patient returns for a later check-up, the headpiece and positioner arrangement can be duplicated. The camera will take the later picture of a skin condition from the same location as the first photographs. Thus changes in the skin condition can be ascertained by comparing the two photographs.

In another embodiment of the invention, the positioning means can be a member that is placed in the patient's mouth. The patient's teeth clamp down on the member, thereby arranging the head in a fixed position. By using a mouthpiece that will make an imprint of the patient's teeth, the position of the device can be replicated at a later visit. In another embodiment, the patient's chin can be placed on a rest. A camera connected to a headpiece or a wall can then be used to photograph the skin for comparison purposes.

The headpiece can be generally ring shaped that is positioned on or about the patient's head. The headpiece can be a single member or can be adjustable so that it fits different sized patients. Similarly, one or both of the positioners may be fixed or one or both may be adjustable for ease of use.

In another embodiment of the invention, the headpiece and/or the positioners are geared to slide open and close proportionally and always keeping the head centered on the device.

In each of the embodiments of the invention there may be additional positioners beyond the ear positioners. These additional positioners may be members extending from the headpiece or secured elsewhere or they may be a laser light pointed to a landmark on the patient, including put not limited to the tip of the nose, the chin or a point on the back of the head.

In each of the embodiments the camera used may be a single camera or multiple cameras positioned appropriately about the head of the patient.

In each of the embodiments the camera used may be placed in fixed positions, on moveable tracks or there may be moveable attachments to secure the camera.

In each of the embodiments the camera may be moved closer to the target lesion for magnifying or may be moved further from the target to increase the field of view.

In each of the embodiments the camera used may be sensitive to visible, ultraviolet, infrared or laser light.

In each of the embodiments the camera may have integrated lighting methods including but not limited to the ones mentioned above which are integrated into the camera, are auto-calibrating and compensate for the ambient lighting.

In each of the embodiments the lighting may be point, ring or field lighting, etc. No matter which arrangement is used, there should be calibrations that can be recorded so that the camera and/or position can be replicated in later examinations, so that the skin condition can be compared from one examination to the next.

### BRIEF DESCRIPTION OF THE DRAWINGS

FIG. 1 shows a perspective view of the positioner of the present invention.

FIG. 2 shows a front view of an alternate embodiment of the device of FIG. 1.

FIG. 3 shows a side view of the embodiment of FIG. 2.

FIG. 4 shows an alternate positioning means for the present invention.

FIG. 5 shows another positioning means of the present invention.

FIG. 6 shows a top view of the mouthpiece of the embodiment of FIG. 4.

FIG. 7 shows a side view of the mouthpiece of FIG. 6.

FIG. 8 shows a camera having an LED ring light and polarized filters.

### DETAILED DESCRIPTION

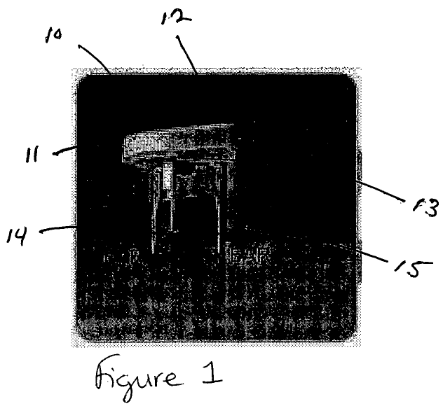

A representative example of a device showing the substance of the positioning apparatus 10 of the present invention is shown in FIG. 1. The device shown in FIG. 1 has a section or headpiece 11 that secures the device to the head of the patient. The headpiece may rest on the top surface of the patient's head. It may have a top surface 12 and a bottom surface 13. The headpiece may be open or closed in its center. If open, the headpiece will rest around the head along the side edges of the patient's head. If closed in the center, the underside can be recessed to receive the top portion of a patient's head. The headpiece may have first 14 and second 15 arms extending therefrom. These arms extend around the side of the patient's head and are preferably slightly flexible so that they spring gently towards the patient's head and hold the device in a secure position. Alternatively, there may be other means of retaining the device on the patient's head such as by means of a strap, belt clasped arm etc. The arms 14 and 15 that come off the device, for example, and extend downwardly for the purpose of positioning the patient's head can be made out of any suitable material. One preferred material is high density opaque thermoplastic material. The ends of the arms that are placed adjacent to the patient's ears such as behind the ears or in the ears. If placed in the ears, the portion of the arms in the patient's ears are made of a suitable soft material which can be disposable or sterilized or covered with a thin disposable sheath to prevent the transfer of germs from one patient to another. One preferred material is natural rubber. The arms are attached the main body of the device which is positioned above the head of the patient. The arms usually extend downwardly from the headpiece and can be stationary or one or both of the arms can slide inwardly and outwardly of the main body and/or frontwardly and backwardly along a track or on a hinge.

In another embodiment of the invention one or both of the ear armatures ride on a track and are geared so that when one is moved toward a desired position they both move proportionally so that the patient's head remains centered in the device.

In another embodiment of the invention both of the ear armatures are hinged and can be raised to allow the patient's head to be positioned readily in the device.

The apparatus of the present invention also includes one or more cameras which record the image of the patient inside the device. The camera can either be attached to the device for example the head piece or separate there from. If attached to the device, it may be secured in an appropriate manner to the head piece. Preferably the camera is movable when attached to the head piece or attached to a wall, etc. Once the patient is positioned inside of the device the doctor activates the device and the cameras begin recording. The camera can be positioned in a fixed location and be moved by the doctor to image different portions of the patient or the camera can be mounted on a movable track which allows the camera to travel around the patient and image everything. Multiple cameras in fixed positions or on tracks can be used also. The camera is capable of zooming in and out to get greater detail or a wider field of vision. The camera may also be sensitive to visible, ultraviolet, infrared or laser light. The camera can have an integrated illumination system which emits the wavelength of illumination which it is sensitive to that is auto-calibrating and compensates for ambient lighting.

When activated the camera may take a single picture or a series of pictures which can be used for a variety of purposes including but not limited to the following. The pictures can be used to generate a printed photograph, or stored on a removable device and given to the patient so that they can compare the lesions (freckles etc.) that they presently have, and if they note any new ones, to advise the physician. The images can be used to create a baseline survey of present lesions and be stored in the patient's records. The images can be digitally modified to improve visibility of the lesions by removing non-lesion artifacts, and overlying hair. The images taken by the doctor can be compared to previously taken stored images of lesions or field of view by using difference or subtraction software to detect any changes or new lesions developing. This allows for more rapid and accurate detection of any changes or new growths versus direct visual inspection. Multiple stored images of a single lesion can be reformatted in 2D and 3D to note and compare specific properties including but not limited to height, contour, crusting, growth rate, asymmetry, abrupt border cutoff, specific colors, presence of pseudopods, radial streaming and scarring. The features of single images can be rapidly compared to image databases such as the DANOS (Diagnostic and Neural Analysis of Skin Cancer) study. A patient can use a home camera to photograph a suspected lesion and send the data to the physician's office to see if it was a previously present at initial screening. Physicians can transmit the image data thru telemedicine for consultations, research projects, second opinions, insurance reimbursement or database additions. Image data can be in standard non-proprietary formats for ease in sharing and analyses or encrypted and non-alterable for patient chart documentation. One program that is particularly useful in the analysis of the photographs is Photoshop and programs like it. These programs have a feature that blends two images and detects any differences.

An alternative embodiment is shown in FIG. 2. There is a wall mounted device 30 which may be used instead of the headpiece of FIG. 1. The wall mounted unit has a mounting means 31 to secure the device to the wall. The mounting means 31 may be a flange or block with screw or nail holes for securing the device to the wall. An arm 32 extends from the mounting means 31 and preferably swivels in at least one plane. Preferably the arm is movable up and down, as well as from side to side. This will permit the technician to adjust the device to fit different size patients.

The arm has a positioning means 33 connected thereto. This positioning means can extend from below the arm or from the end thereof. The positioning means 33 has a first side member 34 and a second side member 35. In the embodiment shown in FIG. 2, the side members are depicted with ear plugs 36 and 37. It will be appreciated that the plugs can be replaced by ear pieces that go behind the ears like eyeglass ear pieces, or ear muff style ear covers or any other means that can go over the ears so that the position of the device on the individual's head can be replicated during later visits. In a preferred embodiment, the ear positioners move in and out simultaneously to always be equidistant from the midline or center of the head.

The side members are preferably adjustable from side to side and/or up and down to accommodate individual head sizes. As shown in FIG. 2, the side piece extends from the arm. The side member has a generally horizontal member 39 that has a first end 40 and a second end 41. At second end 41 there is a vertical member 42 which extends downwardly from the horizontal member 39. The vertical member has a first end 43 and a second end 44. First end 43 connects to second end 41 of the horizontal member 39. At second end 44 of the vertical member 42 there is a second generally horizontal member 45 which may be generally parallel to the first horizontal member 39. The second horizontal member may be provided with a suitable positioning means such as ear plugs 36 and 37.

The first horizontal member in each side member may be adjustable inwardly and outwardly to accommodate heads of different widths. The vertical members may also be adjustable to accommodate the different distances from the top of a patient's head to, for example, the location where the ear plugs are received. Each of the horizontal and vertical members may be calibrated as is the arm and the mounting means so that a reference point can be determined when a reading is taken of a patient so that the same position can be achieved in a later examination.

Besides the side members, there may also be a front member 38 that can be used to align the device with the patient's nose. This front member may be concave so that the nose may be positioned in a portion thereof. Alternatively, the front member may just be a generally vertical member that the nose is aligned with. The front member 38 may have a generally horizontal member 39 extending inwardly that touches the nose or bridge of the nose. A tip 40 can also be present on horizontal member 39. The front member can also be a T shaped member that lines up with the bridge of the nose, the mouth, or the eyes. In one embodiment, the location of the pupils of the eyes can be used when the patient is looking forward. A light means can be used to identify the position of the eyes in this embodiment.

In each of these instances there may be calibrations so that the position of the patient in the first analysis can be replicated in the second visit so that an accurate comparison of the photographs can be achieved.

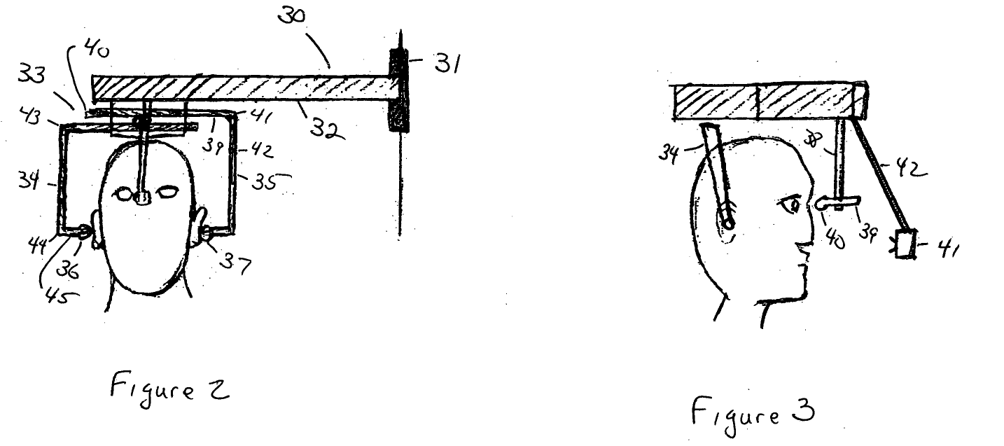

A camera 41 may be mounted on the arm or separately on the wall so that photographs of the patient may be taken. These photographs may be traditional photographs, digital photographs or x-ray photography, infrared photography, etc. The camera may rotate above the patient so that a full 360° view or parts thereof may be taken. Alternatively, the camera may be stationary and the patient may rotate. As seen in FIG. 3, the camera extends from the arm and is positioned to take a picture of the front of the patient. The camera is shown on a leg 42 that extends downwardly from the arm. The height of the camera on the leg can be adjustable if desired. The leg may also have a generally horizontal member that is circular, semicircular or an arc on which the camera can travel to take pictures of different locations on the patient's face.

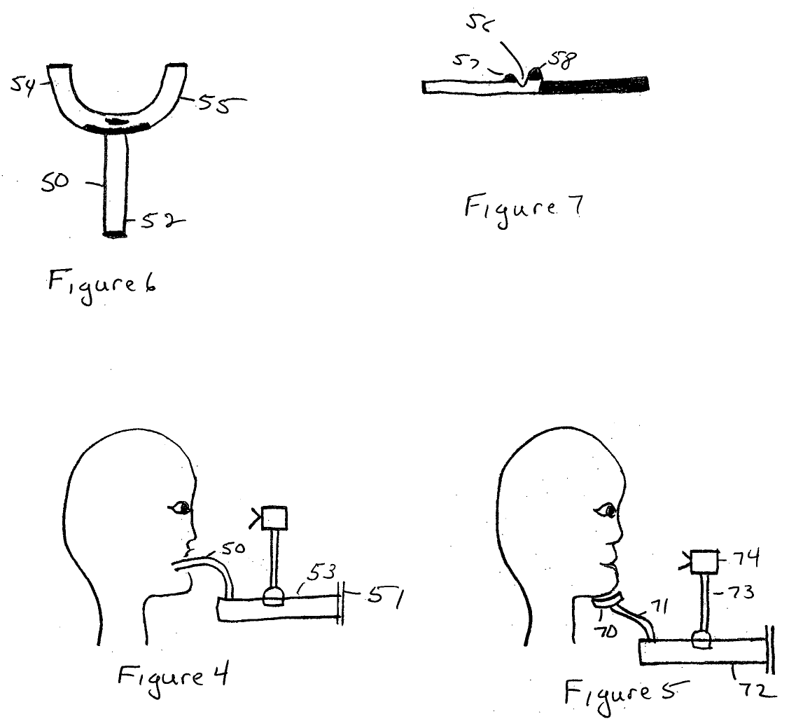

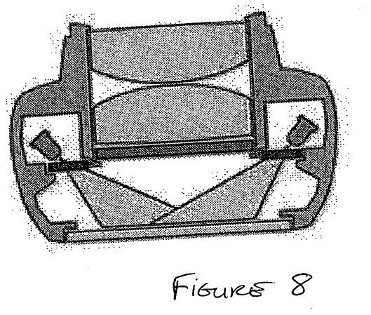

FIG. 4 shows an alternate embodiment where the position of the head is set by a mouthpiece 50. The patient places the mouthpiece in the mouth so that a fixed position is obtained. The fixed position can be replicated at a later date when the patient comes for another visit by using the same mouthpiece. The camera is able to be repositioned to the sides or close up. This is accomplished by using one or more tracks. The end of the base 51 is secured to a wall or desktop floor or to an arm which is secured to the wall or floor. FIG. 6 shows a representation of a sample mouthpiece. The mouthpiece has a stem 52 that may be secured to the camera support 53 by a suitable means. The stem connects the mouthpiece to the main device. Extending from the stem are first 54 and second 55 arms. The arms are preferably generally horizontal and stabilize the head vertically. The arms are preferably generally “u” shaped and provide intraoral positioning. As seen in FIG. 7, the arms have a recessed section 56 and a pair of raised portions 57 and 58. The recessed portion 56 receives the tooth of the patient. The recessed portion 56 on the notch receives the patient's maxillary anterior incisors or other teeth. The patient bites into a plastic material until the maxillary incisors go into the notch.

FIG. 5 shows a chin rest 70 for stabilizing the patient's head so that reproducible photographs can be taken. The chin rest 70 has an arm 71 that extends from a base arm 72. The base arm 72 extends from a wall surface where it is secured thereto. Also extending from the base arm is a camera stand 73 on which a camera 74 is positioned.

FIG. 8 shows a representation of a camera having an LED ring light 80 and polarized filters to emphasize subepithelial characteristics. In FIG. 8 light from the LED passes through the ring polarizer and illuminates the area of examination. The skin absorbs part of the light whereas the reflected light is then cross polarized before returning to the eye of the camera. By filtering out the surface reflection, the technician is able to see deeper structures of skin lesions.

I claim:

1. An apparatus for examining patients for nevi comprising a head piece, said headpiece having at least one positioning means for positioning a patient's head in a reproducible position from one examination to another, said head piece further comprising a camera that is connected to said head piece that can take a picture of at least a portion of a patient's head at a first examination and substantially the same portion of a patient's head at a second examination and whereby the position of the camera can be set so that the picture taken during the first examination can be accurately compared to the picture taken during the second examination.

2. The apparatus according to claim 1 wherein said positioning means is a side member extending from said head piece and aligned with a feature on a patient's head so that it provides a reference point fro aligning the camera subsequent examinations.

3. The apparatus according to claim 3 wherein there is a first positioning member and a second positioning member, said first and second positioning members being arranged to be aligned with the ears of a patient.

4. The apparatus according to claim 3 wherein said first and second positioning members have plugs for insertion into a portion of a patient's ear.

5. The apparatus according to claim 2 wherein said positioning member is arranged to be aligned with a patient's nose.

6. An apparatus for examining patient's for nevi comprising a means for positioning a patient's head in a first position so that a photograph of the patient's skin may be3 taken, said first position being reproducible in a second examination so that a photograph taken during the second examination can be accurately compared to the photograph taken during the first examination so that changes in skin condition can be determined.

7. The apparatus according to claim 6 wherein said means for positioning comprises a mouthpiece.

8. The apparatus according to claim 6 wherein said means for positioning comprises a chin rest.

9. The apparatus according to claim 7 wherein said mouthpiece has a stem and first and second arms extending from said mouthpiece, said first and second arms being aligned with a patient's teeth when said mouthpiece is in a patient's mouth.

10. The apparatus according to claim 9 wherein said mouthpiece has one or more recesses for aligning a patient's teeth to said mouthpiece.

11. The apparatus according to claim 3 wherein the positioning members move into and out of position simultaneously so that they are always equidistant from a midline.
